(19) United States
(12) Patent Application Publication
(10) Pub. No.: US 2026/0140124 A1
(43) Pub. Date: May 21, 2026

(54) PCOS STRATIFICATION

(71) Applicant: THE UNIVERSITY OF BIRMINGHAM, Birmingham, West Midlands (GB)

(72) Inventor: Wiebke ARLT, London (GB)

(73) Assignee: THE UNIVERSITY OF BIRMINGHAM, Birmingham, West Midlands (GB)

(21) Appl. No.: 19/122,432

(22) PCT Filed: Oct. 23, 2023
(86) PCT No.: PCT/GB2023/052768

§ 371 Date: Apr. 18, 2025

(30) Foreign Application Priority Data
Oct. 21, 2022 (GB) 2215661.6

(51) Int. Cl. G01N 33/74

(52) U.S. Cl. CPC G01N 33/743; G01N 2333/575; G01N 2800/367; G01N 2800/52

(57) ABSTRACT

The present invention provides a method of stratifying test subjects with Polycystic Ovary Syndrome (PCOS) into those that are expected to respond to therapeutic treatment with an inhibitor of hypothalamic-pituitary-adrenal axis activity, those that are expected to respond to therapeutic treatment with an AKR1C3 inhibitor, and those that are expected to respond to therapeutic treatment with a 5alpha-reductase inhibitor, the method comprising: (a) providing a test sample from a test subject with PCOS; (b) determining the concentration/level in the test sample of at least one ovarian androgen steroid and/or a derivative thereof, and at least one adrenal steroid and/or derivative thereof; and (c) using the results from (b) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

### FIELD OF THE INVENTION

The invention relates to a method of stratifying test subjects with Polycystic Ovary Syndrome (PCOS). The invention also relates to a computer system and a computer program for implementing the method according to the invention.

### BACKGROUND OF THE INVENTION

Polycystic ovary syndrome (PCOS) affects at least 10% of women worldwide. PCOS is diagnosed based on the presence of clinical and/or biochemical signs of androgen excess, chronic anovulation/irregular menstrual cycles, and polycystic appearance of the ovaries on ultrasound. According to the widely accepted Rotterdam criteria, at least two out of those three criteria need to be present to make a diagnosis of PCOS, in addition to exclusion of other rare possible underlying causes.

PCOS was previously mainly perceived as a disorder affecting women of reproductive age and the by far most common cause of female infertility. However, over recent years it has become clear that PCOS is a lifelong metabolic disorder and multiple studies have demonstrated that women with PCOS have a 2-3-fold increased risk of type 2 diabetes, hypertension, fatty liver disease and cardiovascular disease. Thus, women with PCOS represent a major risk group for the development of metabolic disease and associated co-morbidities and mortality. However, due to the large number of affected women, health care systems struggle to manage this risk effectively, and there are currently no tools for metabolic risk stratification, an urgent unmet clinical need. The present invention seeks to address these problems.

### STATEMENTS OF THE INVENTION

The inventors believe but do not wish to be bound by the theory that an increase of steroid hormones, particularly androgens, in women with PCOS is responsible for causing PCOS and metabolic disorders that are known to be associated with PCOS. However, in some women with PCOS, there is also an increase in the ratio of the concentration/level of hormones synthesised in the adrenal glands compared to the concentration/level of hormones synthesised in the ovaries e.g., ovarian androgens. The inventors believe but do not wish to be bound by the theory that this relative increase in the concentration/level of hormones synthesised in the adrenal glands is indicative of whether a subject will respond to certain types of therapy for PCOS and any metabolic disorders that may be associated with PCOS.

Thus, according to a first aspect of the invention, there is provided a method of stratifying test subjects with Polycystic Ovary Syndrome (PCOS) into those that are expected to respond to therapeutic treatment with an inhibitor of hypothalamic-pituitary-adrenal axis activity, those that are expected to respond to therapeutic treatment with an AKR1C3 inhibitor, and those that are expected to respond to therapeutic treatment with a 5alpha-reductase inhibitor, the method comprising:
(a) providing a test sample from a test subject with PCOS;
(b) determining the concentration/level in the test sample of at least one ovarian androgen steroid and/or a derivative thereof, and at least one adrenal steroid and/or derivative thereof; and
(c) using the results from (b) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

Step (b) (i.e., determining the concentration/level in the test sample of at least one ovarian androgen steroid or derivative thereof and at least one adrenal androgen steroid or derivative thereof) may comprise determining the concentration/level in the test sample of at least one, at least two, at least three, or at least four ovarian androgen steroids or derivatives thereof.

The ovarian androgen steroids or derivatives thereof may be one or more, two or more, or three or more selected from the group comprising androstenedione (Adione), testosterone, 5α-dihydrotestosterone (DHT) and 5α-androstanedione (5Adione). The ovarian androgen steroids or derivatives thereof may further comprise androsterone (An). Thus, the ovarian androgen steroids or derivatives thereof may comprise Adione, Testosterone, DHT, 5adione and An.

In embodiments with at least one ovarian androgen steroid or derivative thereof, the ovarian androgen steroid may be or comprise DHT. In embodiments with at least four ovarian androgen steroids or derivatives thereof, the steroids may be Adione, testosterone, DHT and 5Adione, and optionally An.

Steroids synthesised in the adrenal glands (adrenal steroids or derivatives thereof) may be divided into three classes: mineralocorticoids, glucocorticoids and sex hormones, including androgens. Step (b) may comprise determining the concentration/level of at least one adrenal steroid. Thus, step (b) may comprise determining the concentration/level of at least one adrenal steroid selected from mineralocorticoids, glucocorticoids and androgens. Preferably the at least one adrenal steroid comprises or consists of adrenal androgen steroids, such as DHEAS, DHEA, Adione, 11OHA4, and 11KA4.

Step (b) may comprise determining the concentration/level in the test sample of at least one, at least two, at least three, at least four, at least five, at least six, at least seven, at least eight, at least nine, at least ten, or at least eleven adrenal steroids or derivatives thereof. The at least one adrenal steroid may be selected from the group comprising dehydroepiandrosterone sulfate (DHEAS), dehydroepiandrosterone (DHEA), 11-hydroxyandrostenedione (11OHA4), 11-ketoandrostenedione (11KA4), 11-ketotestosterone (11KT), 11-hydroxytestosterone (11OHT), pregnenolone, 17-hydroxypregnenolone, 17-hydroxyprogesterone (17OHP), 11-deoxycortisol (S), cortisol (F), d11-deoxycorticosterone (DOC), corticosterone (B) and aldosterone (Aldo).

The at least one adrenal steroid may be one or more, two or more, three or more, four or more, five or more, six or more, seven or more, eight or more, nine or more, 10 or more or 11 or more selected from the group comprising dehydroepiandrosterone sulfate (DHEAS), dehydroepiandrosterone (DHEA), 11-hydroxyandrostenedione (11OHA4), 11-ketoandrostenedione (11KA4), 11-ketotestosterone (11KT), 11-hydroxytestosterone (11OHT), pregnenolone, 17-hdyroxypregnenolone, 17-hydroxyprogesterone (17OHP), 11-deoxycortisol (S), cortisol (F), d11-deoxycorticosterone (DOC), corticosterone (B) and aldosterone (Aldo).

Preferably step (b) comprises determining the concentration/level in the test sample of at least six adrenal steroids or derivatives thereof. The at least six adrenal steroids or derivatives thereof may be dehydroepiandrosterone sulfate (DHEAS), dehydroepiandrosterone (DHEA), 11-hydroxyandrostenedione (11OHA4), 11-ketoandrostenedione (11KA4), 11-ketotestosterone (11KT) and 11-hydroxytestosterone (11OHT). The at least six or more adrenal steroids or derivatives thereof may be selected from the group comprising pregnenolone, 17-hydroxypregnenolone, DHEA, 11KT, 17-hydroxyprogesterone (17OHP), 11-deoxycortisol (S), cortisol (F), d11-deoxycorticosterone (DOC), corticosterone (B) and aldosterone (Aldo).

In one embodiment, step (b) comprises determining the concentration/level in the test sample of at least one ovarian androgen steroid and/or a derivative thereof, and at least six adrenal steroids and/or derivatives thereof. Preferably the ovarian androgen steroid or derivative thereof is DHT, and the adrenal steroids or derivatives thereof are pregnenolone, 17-hydroxypregnenolone, DHEA, 11KT, 17OHP, S, F, DOC, B and Aldo.

In one embodiment, step (b) comprises determining the concentration/level in the test sample of at least four ovarian androgen steroids or derivatives thereof and the concentration/level in the test sample of at least six adrenal steroids or derivatives thereof. Preferably the ovarian androgen steroids or derivatives thereof are Adione, Test, DHT and 5Adione, and the adrenal steroids or derivatives thereof are DHEAS, DHEA, 11OHA4, 11KA4, 11KT and 11OHT.

In one embodiment, the method may further comprise comparing the concentrations/levels determined in (b) with reference concentrations of the hormones and/or their derivatives thereof from other subjects with PCOS. The method may further comprise using the results from (c) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

Thus, in one embodiment, the method comprises:
(a) providing a test sample from a test subject with PCOS;
(b) determining the concentration/level in the test sample of at least one ovarian androgen steroid and/or a derivative thereof, and at least one adrenal steroid and/or derivative thereof;
(c) comparing the concentrations/levels determined in (b) with reference concentrations of the hormones and/or their derivatives thereof from other subjects with PCOS; and
(d) using the results from (c) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

In one embodiment, the method comprises:
(a) providing a test sample from a test subject with PCOS;
(b) determining the concentration/level in the test sample of at least one ovarian androgen steroid and/or a derivative thereof, and at least six adrenal steroids and/or derivatives thereof;
(c) comparing the concentrations/levels determined in (b) with reference concentrations of the hormones and/or their derivatives thereof from other subjects with PCOS; and
(d) using the results from (c) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

According to a second aspect, there is provided a method of stratifying test subjects with PCOS into those that are expected to respond to therapeutic treatment with an inhibitor of hypothalamic-pituitary-adrenal axis activity, those that are expected to respond to therapeutic treatment with an AKR1C3 inhibitor, and those that are expected to respond to therapeutic treatment with a 5alpha-reductase inhibitor, the method comprising:
(a) providing a test sample from a test subject with PCOS;
(b) determining the concentration/level in the test sample of at least one ovarian androgen steroid or derivative thereof comprising DHT, and at least six adrenal steroids and/or derivatives thereof comprising pregnenolone, 17-hydroxypregnenolone, DHEA, 11KT, 17OHP, S, F, DOC, B and Aldo; and
(c) using the results from (b) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

Step (b) (i.e., determining the concentration/level in the test sample of at least one ovarian androgen steroids or derivatives thereof and at least six adrenal steroids or derivatives thereof) may comprise determining the concentration/level in the test sample of at least one, at least two, or at least three ovarian androgen steroids or derivatives thereof (e.g., DHT and testosterone and/or An). Thus, step (b) may further comprise determining the concentration/level in the test sample of testosterone and/or An.

Step (b) may further comprise determining the concentration/level in the test sample of one or more adrenal steroids and/or the derivatives selected from the group comprising DHEAS, Adione, 11OHA4 and 11KA4.

Step (b) may further comprise determining the concentration/level in the test sample of one or more ovarian androgen steroids and/or the derivatives selected from the group comprising Adione, 5adione, testosterone and An. Step (b) may further comprise determining the concentration/level in the test sample of one or more ovarian androgen steroids and/or the derivatives selected from the group comprising 5adione, testosterone and An.

Step (b) may further comprise determining the concentration/level in the test sample of 11OHT and/or E.

In one embodiment, step (b) comprises determining the concentration/level in the test sample of at least 20 steroids comprising Preg, 17Preg, DHEAS, DHEA, Adione, testosterone, 5adione, An, 11OHA4, 11KA4, 11Kt, 11OHt, 17OHP, S, F, E, DOC, B and Aldo.

In one embodiment, the method comprises comparing the concentrations/levels determined in (b) with reference concentrations of the steroids and/or their derivatives thereof from other subjects with PCOS. The embodiment may further comprise the step of using the results to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

Thus, in one embodiment, the method comprises:
(a) providing a test sample from a test subject with PCOS;
(b) determining the concentration/level in the test sample of at least one ovarian androgen steroid or derivative thereof comprising DHT, and at least six adrenal steroids and/or derivatives thereof comprising pregnenolone, 17-hydroxypregnenolone, DHEA, 11KT, 17OHP, S, F, DOC, B and Aldo;
(c) comparing the concentrations/levels determined in (b) with reference concentrations of the steroids and/or their derivatives thereof from other subjects with PCOS; and
(d) using the results from (c) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

According to a third aspect, there is provided a method of stratifying test subjects with PCOS into those that are expected to respond to therapeutic treatment with an inhibitor of hypothalamic-pituitary-adrenal axis activity, those that are expected to respond to therapeutic treatment with an AKR1C3 inhibitor, and those that are expected to respond to therapeutic treatment with a 5alpha-reductase inhibitor, the method comprising:
(a) providing a test sample from a test subject with PCOS;
(b) determining the concentration/level in the test sample of at least three ovarian androgen steroids and/or derivatives thereof comprising testosterone, DHT and 5adione, and at least six adrenal steroids and/or derivatives thereof comprising DHEAS, DHEA, 11OHA4, 11KA4, 11KT and 11OHT; and
(c) using the results from (b) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

Step (b) (i.e., determining the concentration/level in the test sample of at least three ovarian androgen steroids or derivatives thereof and at least six adrenal steroids or derivatives thereof) may comprise determining the concentration/level in the test sample of at least three, at least four, or at least five ovarian androgen steroids or derivatives thereof. For example, step (b) may comprise determining the concentration/level in the test sample of at least four ovarian androgen steroids or derivatives thereof comprising Adione, testosterone, DHT, and 5adione, and optionally An. Step (b) may comprise determining the concentration/level in the test sample of at least six, at least seven or at least eight adrenal steroids or derivatives thereof. Therefore, step (b) may comprise determining the concentration/level in the test sample of at least three, at least four, or at least five ovarian androgen steroids or derivatives thereof, and at least six, at least seven, at least eight, at least nine, at least ten, or at least 11 adrenal steroids or derivatives thereof. In one embodiment, step (b) comprises determining the concentration/level in the test sample of at least four ovarian androgen steroids or derivatives thereof comprising Adione, testosterone, DHT, 5adione, and optionally An, together with at least six adrenal steroids or derivatives thereof comprising DHEAS, DHEA, 11OHA4, 11KA4, 11KT and 11OHT.

In one embodiment, step (b) comprises determining the concentration/level in the test sample of at least four ovarian androgen steroids and/or derivatives thereof comprising adione, testosterone, DHT and 5adione, and at least six adrenal steroids and/or derivatives thereof comprising DHEAS, DHEA, 11OHA4, 11KA4, 11KT and 11OHT. In one embodiment, step (b) comprises determining the concentration/level in the test sample of at least four ovarian androgen steroids and/or derivatives thereof comprising An, testosterone, DHT and 5adione, and at least six adrenal steroids and/or derivatives thereof comprising DHEAS, DHEA, 11OHA4, 11KA4, 11KT and 11OHT.

In one embodiment, step (b) comprises determining the concentration/level in the test sample of at least six adrenal steroids or derivatives thereof comprising DHEAS, DHEA, 11OHA4, 11KA4, 11KT and 11OHT.

In one embodiment, step (b) comprises determining the concentration/level in the test sample of at least 10 steroids comprising DHEAS, DHEA, Adione, testosterone, DHT, 5adione, 11OHA4, 11KA4, 11KT and 11OHT.

In one embodiment, step (b) comprises determining the concentration/level in the test sample of at least 11 steroids comprising DHEAS, DHEA, Adione, testosterone, DHT, 5adione, An, 11OHA4, 11KA4, 11KT and 11OHT.

In one embodiment, the method comprises comparing the concentrations/levels determined in (b) with reference concentrations of the steroids and/or their derivatives thereof from other subjects with PCOS. The embodiment may further comprise the step of using the results to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

Thus, in one embodiment, the method comprises:
(a) providing a test sample from a test subject with PCOS;
(b) determining the concentration/level in the test sample of at least three ovarian androgen steroids and/or derivatives thereof comprising testosterone, DHT and 5adione (and optionally Adione and/or An), and at least six adrenal steroids and/or derivatives thereof comprising DHEAS, DHEA, 11OHA4, 11KA4, 11KT and 11OHT;
(c) comparing the concentrations/levels determined in (b) with reference concentrations of the steroids and/or their derivatives thereof from other subjects with PCOS; and
(d) using the results from (c) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

The step of comparing the concentrations/levels determined in (b) with reference concentrations of the steroids and their derivatives thereof from other subjects with PCOS recited herein may comprise performing a cluster analysis.

The steroids thereof recited herein may be or comprise active steroids. The adrenal steroids may be or comprise active steroids. Active steroids can refer to steroids that are capable of binding to a steroid receptor, e.g., the androgen receptor. Active adrenal steroids include 11-ketotesterone, 11-hydroxyteststerone, cortisol and aldosterone. The ovarian steroids (e.g., ovarian androgen hormones) may be or comprise active steroids. Active ovarian steroids include testosterone and DHT.

The inventors believe, but do not wish to be bound by the hypothesis that individuals with PCOS are not a homogeneous cohort but consist of many different conditions that present with a similar phenotype of androgen excess, irregular cycles and increased metabolic risk. Advantageously, the method according to the invention allows subjects with PCOS to be provided with the most appropriate treatment based on their steroid levels/concentrations Thus, the invention allows one to identify subjects with PCOS who are at a higher risk of developing a metabolic disorder. Consequently, one can implement a more intense monitoring strategy as part of their management, for prevention, early detection and treatment of metabolic disease including those occurring in pregnancy, such as gestational diabetes and pregnancy-related hypertension, conditions more prevalent in women with PCOS. Thus, the invention enables a clinician to ensure that each subject receives the best course of treatment for their PCOS and any associated metabolic disorder. The invention will therefore enables clinicians to reduce the amount of time and money wasted on providing subject with inappropriate treatment.

It will be appreciated that determining the concentration/level in a test sample of a steroid may be performed determined using any suitable method/technique/technology known in the art. Preferably, the step of determining the concentration/level in a test sample of a steroid may be performed determined using any suitable method/technique/technology known in the art comprises mass spectrometry.

The inhibitors referred to herein (i.e., inhibitors of the hypothalamic-pituitary-adrenal axis activity, AKR1C3 inhibitors, 5alpha-reductase inhibitors) may be used to treat PCOS only, a metabolic disorder associated with PCOS, or PCOS and an associated metabolic disorder. Inhibitors or modifiers of hypothalamic-pituitary-adrenal axis activity include modified release glucocorticoids or antagonists of corticotrophin-releasing hormone (CRH).

An AKR1C3 inhibitor includes any agent that is capable of inhibiting the enzymatic activity of AKR1C3. AKR1C3 is the enzyme that converts androstenedione to testosterone and androstenedione to 11-hydroxyandrostenedione. AKR1C3 is the only tissue in adipose tissue that can activate androgens and AKR1C3 converts androstenedione to 11-hydroxyandrostenedione 10-fold faster than it converts androstenedione to testosterone. Thus, AKR1C3 inhibition specifically reduces 11-oxygenated androgen excess, both systemically and in adipose tissue.

A 5α-reductase inhibitor is an agent that inhibits the enzyme activity of 5α-reductase (e.g., finasteride, dutasteride). 5α-reductase catalyses the activation of T to DHT, which is more potent than T. Therefore, inhibiting the enzyme can dampen down androgen activity.

According to another aspect of the invention, there is provided a computer system having a processor, a memory and an input for receiving data obtained from a method according to the invention (e.g., the data of obtained in step (b) and/or step (c)), the computer configured to use the data obtained from the method according to the invention (as mentioned in step (c) or (d)), and having a computer program adapted to perform the method of the invention.

According to another aspect, there is provided a computer program comprising instructions which, when run on a computer with a processor, memory and input, to configure it to the computer system according to the invention.

The subject is preferably a mammal, in particular a human, but it may also be an animal, such as a dog, cat, horse, cow, sheep or pig. Thus, the subject may be a human subject. The subject may be a subject with PCOS and a metabolic disorder, such as diabetes (Type I and/or type II). Preferably, the subject is human with PCOS and type 2 diabetes, hypertension, fatty liver disease and/or cardiovascular disease. Most preferably, the subject is a human with PCOS and type 2 diabetes.

Subjects with high adrenal steroids levels/concentrations are expected to respond to treatment with an inhibitor of hypothalamic-pituitary-adrenal axis activity and/or a AKR1C3 inhibitor. Subjects with high ovarian steroids levels/concentrations, particularly DHT, are expected to respond to treatment with a 5alpha-reductase inhibitor.

The test sample is preferably a sample taken from the body of the test subject. The sample may be or comprise blood, plasma, or serum. Thus, determining the concentration/level in the test sample is therefore preferably carried out in vitro.

Step (a) of the invention may be optional. Thus, the step (a) may comprise determining the concentration/level in a test sample that has been obtained from a test subject with PCOS.

The method according to the invention may comprise comparing the concentrations/levels determined in (b) with reference concentrations of the steroids and/or their derivatives thereof from “n” other subjects with PCOS. It will be appreciated that the larger that “n” is, the more accurate the method according to the invention will be. Thus, “n” may be at least 5 or more, at least 10 or more, at least 20 or more, at least 30 or more, at least 40 or more, at least 50 or more, at least 100 or more, at least 150 or more, at least 200 or more, at least 250 or more, at least 300 or more, at least 300 or more, at least 400 or more, or at least 500 or more other subjects with PCOS. Preferably the other subjects have not been treated for PCOS as this keeps confounding factors to a minimum.

The method according to the invention may be computer implemented.

The term “ovarian androgen steroid” or “ovarian androgen” refers to androgenic steroids synthesised mainly in the ovary. The term “adrenal steroids” refers to steroids synthesised in the adrenal gland.

TABLE 1
discloses steroids that may be used to perform
the method according to the invention

| Hormone | Full name | Class | Location of synthesis |
|---|---|---|---|
| Preg | Pregnenolone | Central precursor | Adrenal and ovary |
| 17Preg | 17-hydroxypregnenolone | Central precursor | Adrenal and ovary |
| DHEAS | DHEA sulphate | Androgen precursor | Adrenal |
| DHEA | Dehydroepiandrosterone | Androgen precursor | Adrenal |
| Adione | Androstenedione | Androgen precursor | Adrenal and ovary |
| Test | Testosterone | Active androgen | Mainly ovary |
| DHT | 5α-dihydrotestosterone | Active androgen | Mainly ovary |
| 5adione | 5α-androstanedione | Androgen metabolite | Mainly ovary |
| An | Androsterone | Androgen metabolite | Mainly ovary |
| 11OHA4 | 11β- | Androgen precursor | Adrenal |
| | hydroxyandrostenedione | | |
| 11KA4 | 11-ketoandrostenedione | Androgen precursor | Adrenal |
| 11KT | 11-ketostosterone | Active androgen | Mainly adipose tissue |
| 11OHT | 11β-hydroxytestosterone | Active androgen | Mainly adipose tissue |
| 17OHP | 17α-hydroxyprogesterone | Central precursor | Adrenal and ovary |
| S | 11-deoxycortisol | Glucocorticoid | Adrenal |
| | | precursor | |
| F | Cortisol | Active | Adrenal |
| | | glucocorticoid | |
| E | Cortisone | Glucocorticoid | Kidney, salivary gland |
| | | metabolite | |
| DOC | 11-deoxycorticosterone | Mineralocorticoid | Adrenal |
| | | precursor | |
| B | Corticosterone | Mineralocorticoid | Adrenal |
| | | precursor | |
| Aldo | Aldosterone | Active | Adrenal |
| | | mineralocorticoid | |

The term “derivatives thereof” can refer to immediate metabolites and/or immediate precursors of active androgens. The term “active androgens” can refer to androgens that bind to the androgen receptor, such as testosterone, DHT, 11KT and 11OHT. The immediate precursor of testosterone is A4. The immediate metabolite of testosterone is DHT. The immediate precursor of DHT is testosterone. The immediate metabolite of testosterone is androsterone. The immediate precursor of 11KT is 11KA4. The immediate metabolite of 11KT is 11-hydroxytestosterone

The skilled person will appreciate how to perform a cluster analysis. This may, for example, be achieved using 3k-means clustering or hierarchical clustering.

3k-means clustering may comprise defining the n-by-p matrix X containing the data. Each row of X represents one of the p samples, each column of X represents one of the n measured metabolite concentrations with e.g. n=11 or n=21. First, a standardization by means of a z-score transformation [1] is applied to the data set: we calculate the z-score for each element of X such that columns of the transformed matrix X are centered to have mean 0 and scaled to have standard deviation 1. Next, an analysis by use of an established clustering method is performed. For instance, k-means clustering is applied [2,3] which represents potential clusters in the data in terms of k n-dimensional vectors (the centroids or means). The following describes a possible implementation of the clustering procedure. For a specific analysis, the algorithm is performed with a fixed number k, e.g., with k=3. The k-means++ algorithm is employed for the cluster center initialization [4] and then the batch version of the standard k-means algorithm is performed [3] using squared Euclidian as the distance metric. Subsequently, the clustering is refined by performing the online version of k-means as outlined in [2] for up to 2000 iterations in order to guarantee reaching a local minimum of the distance criterion. Run 1000 replicates are then run, returning the clustering with the lowest within-cluster sums of point-to-centroid distances.

The term “comprising” can refer to “consisting of”.

All of the embodiments and features described herein (including any accompanying claims, abstract and drawings), and/or all of the steps of any method or process so disclosed, may be combined with any of the above aspects or embodiments in any combination, unless stated otherwise with reference to a specific combination, for example, combinations where at least some of such features and/or steps are mutually exclusive.

### FIGURES

For a better understanding of the invention, and to show embodiments of the invention may be put into effect, reference will now be made, by way of example, to the accompanying drawings, in which:—

FIG. 1 shows the results of a 3k-means clustering of the steroid metabolome measured by mass spectrometry-based multi-steroid profiling. Two data sets were used, one consisting of 11 androgenic steroids (left panel), the other of 20 steroids covering androgens, glucocorticoids and mineralocorticoids (right panel). Each point represents one steroid metabolome (=one patient with PCOS) visualised by PCA and hence projected according to the first two principal components. All patients fall into one of three distinct clusters (marked blue, red and green—the blue cluster is on the left; the green cluster is on the right and the red cluster is in the middle), which have hardly any overlap;

FIG. 2 is a summary of the data consisting of 11 androgenic steroids. For each steroid in the bar graph, the red bar (corresponding to the red cluster) is first displayed, followed by a green bar (corresponding to the green cluster) and then a blue bar (corresponding to the blue cluster);

FIG. 3 is a summary of the data consisting of the 20 steroids covering androgens, glucocorticoids and mineralocorticoids. For each steroid in the bar graph, the red bar (corresponding to the red cluster) is first displayed, followed by a green bar (corresponding to the green cluster) and then a blue bar (corresponding to the blue cluster);

FIG. 4 shows the results of a cluster analysis performed on an 11-steroid cluster (left panel) and a 20-steroid cluster (right panel) with the steroid metabolomes of the healthy controls (indicated in yellow) overlaying the left-hand cluster in both the left panel and the right panel—the blue cluster is on the left; the green cluster is on the right and the red cluster is in the middle;

FIG. 5 shows the steroid synthesis pathway for mineralocorticoids, glucocorticoids and androgens;

FIG. 6 shows the steroid synthesis pathway for mineralocorticoids, glucocorticoids and androgens, and includes the 11-oxygenated androgen pathway;

FIG. 7 shows a summary of the classical androgen biosynthesis pathway, the 11-oxygenated androgen pathway, and the alternative DHT biosynthesis pathway;

FIG. 8 shows a summary of the classic androgen synthesis pathway and the 11-oxygenated androgen pathway;

FIG. 9 is a box plot that shows the differences in the Homeostatic Model Assessment for Insulin Resistance between the three 11-steroid clusters; and

FIG. 10 is a box plot that shows the differences in the Insulin Sensitivity Index between the three 11-steroid clusters.

### EXAMPLES

The inventors believe that individuals with PCOS are not a homogeneous population. Rather, they are population that consists of individuals with several different conditions that present with a similar phenotype of androgen excess, irregular cycles and increased metabolic risk that may be manifest in variable degrees.

The inventors performed steroid metabolome profiling with subsequent unsupervised cluster analysis to identify distinct steroid phenotypes in PCO patient groups and related this information to their clinical phenotypes in order to stratify them according to the type of therapy that they should receive.

This novel approach was performed using serum collected from 500 patients with confirmed PCOS by mass spectrometry-based steroid profiling. The data has been subjected to unsupervised clustering by 3k-means clustering, followed by visualisation of the multidimensional data by principal component analysis (PCA). This was performed with two datasets: first, the entire 20 steroids measured in the multi-steroid profile, second, with only those 11 out of 20 steroids that are either active androgens or immediate precursors or immediate metabolites of active androgens.

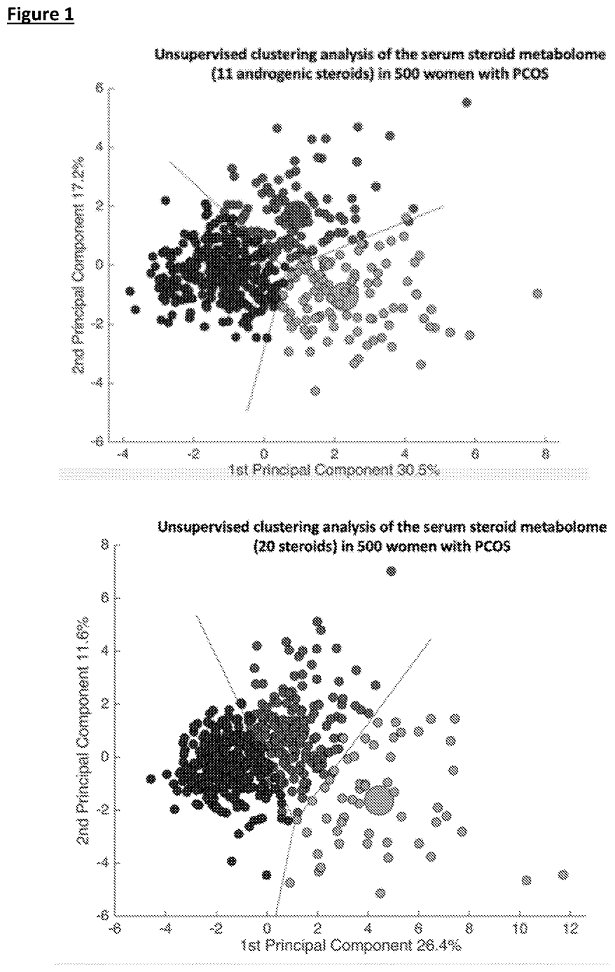

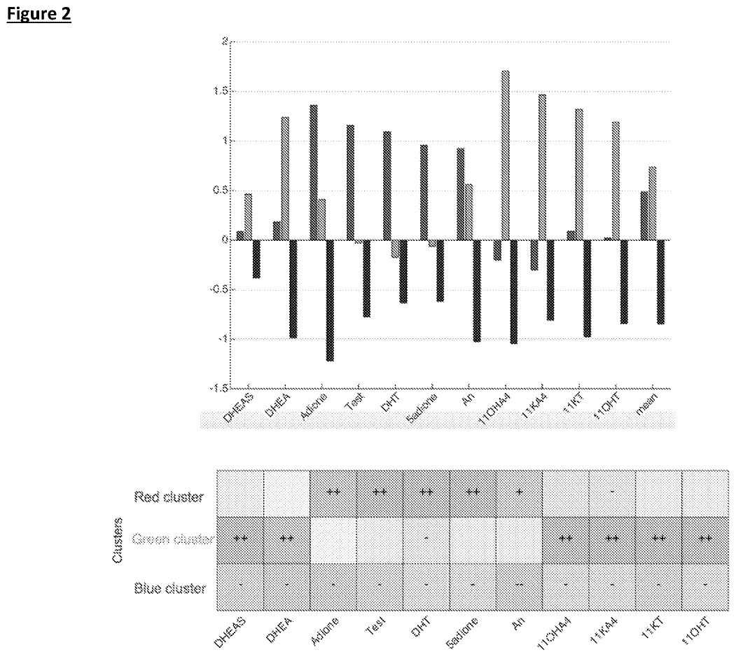

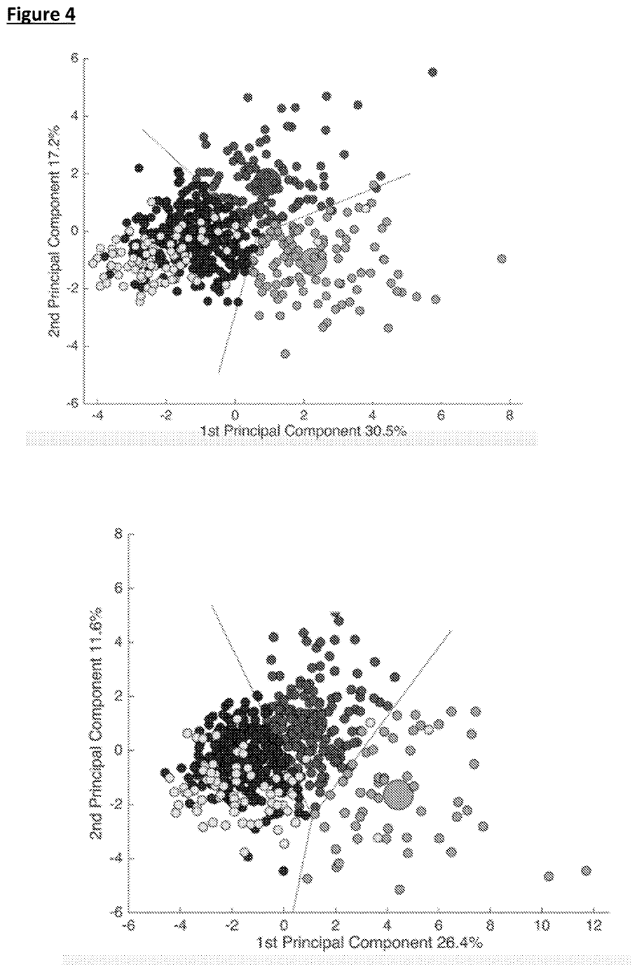

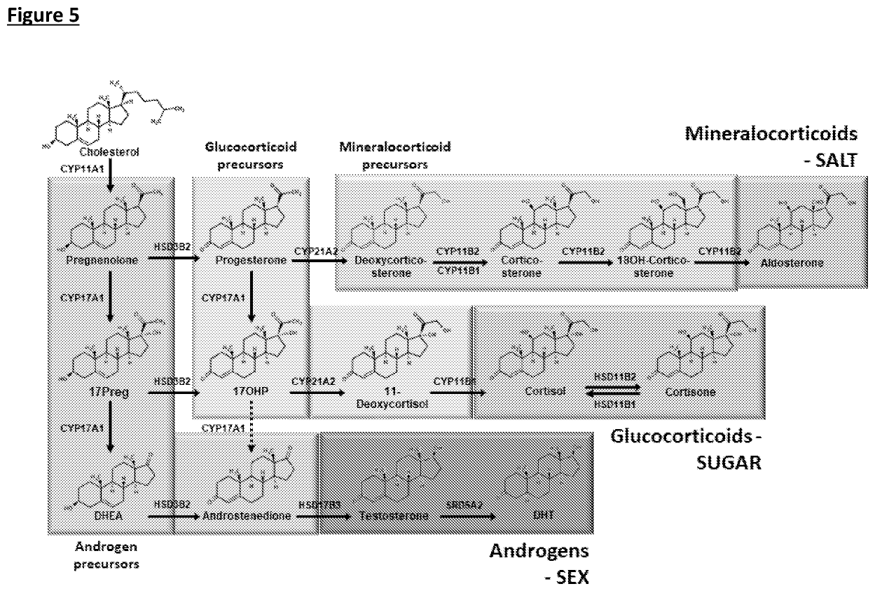

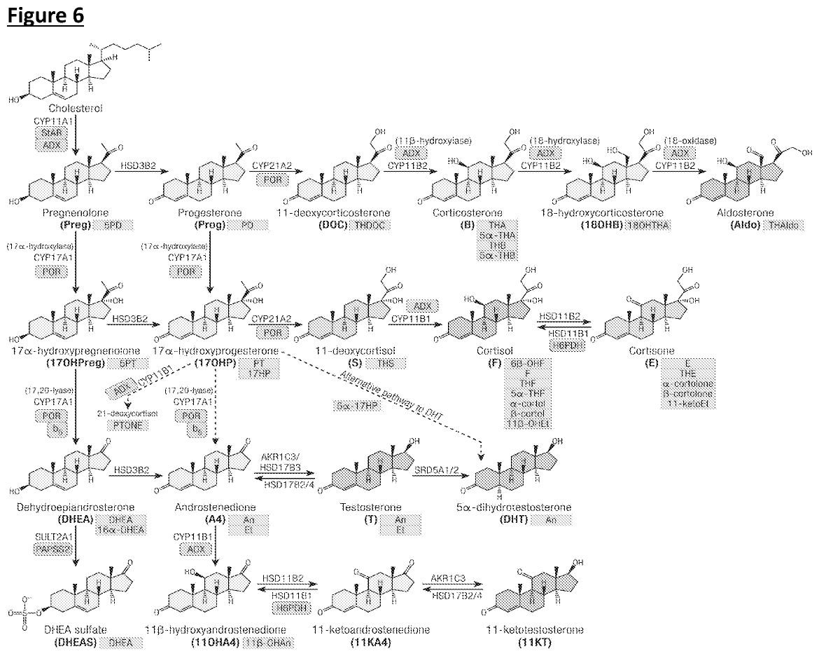

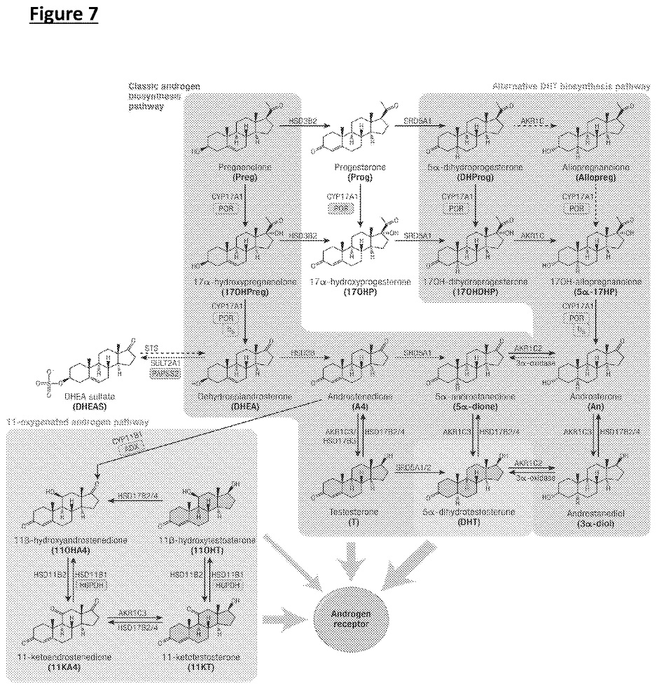

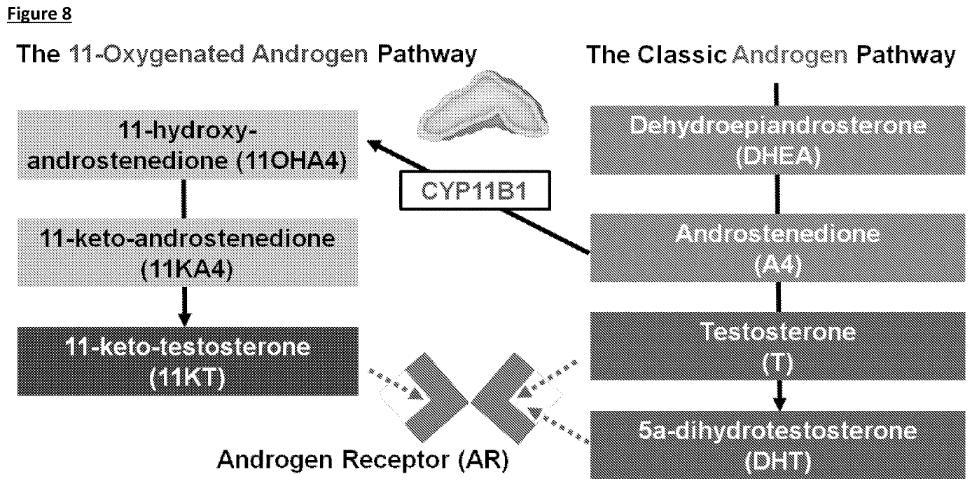

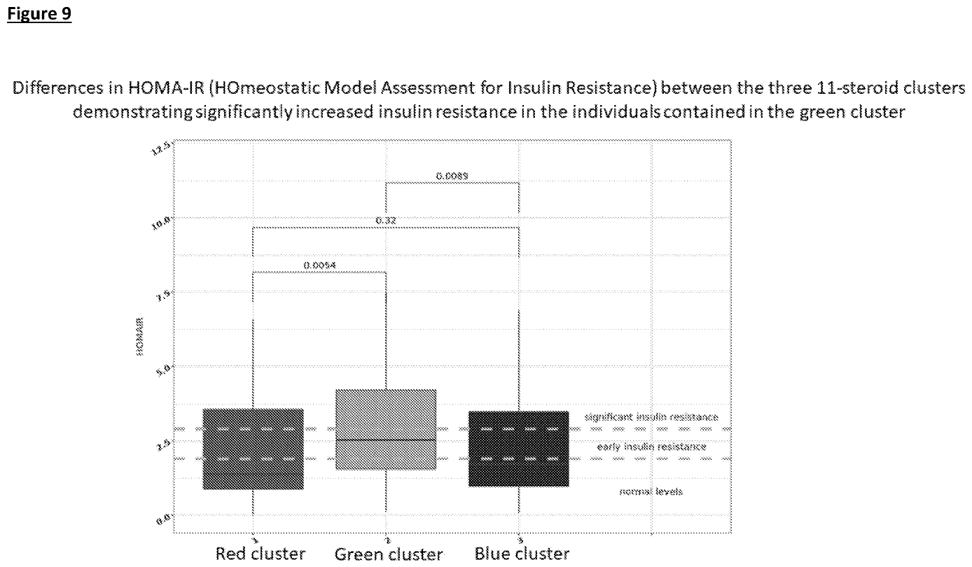

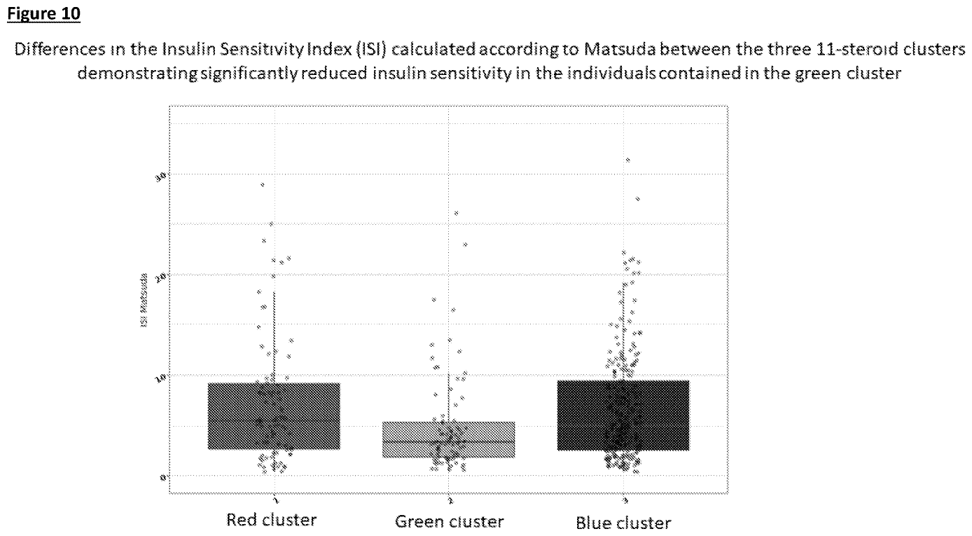

The approach mentioned above surprisingly and unexpectedly resulted in the PCOS cohort being divided into three distinct clusters based on their steroid phenotype (see FIGS. 1 and 4—the blue cluster is on the left; the green cluster is on the right and the red cluster is in the middle). The inventors have confirmed that these clusters are very stable and can be reproduced in principle also when using other clustering techniques, such as hierarchical clustering.

### Example 1—Clustering Based on 11 Steroids

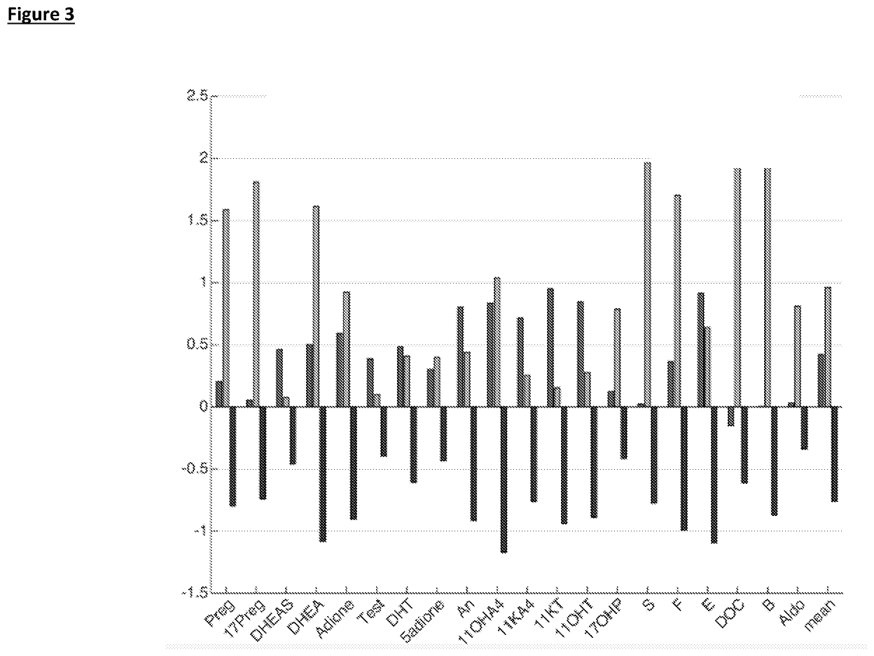

When analysing which steroids inform the differences between the three steroid clusters, a very interesting picture emerged. In the clustering with the 11 androgenic steroids, the blue cluster was characterised by generally lower levels, while the red cluster was characterised by higher classic androgen concentrations (A4, T and DHT) and the green cluster by higher concentrations of adrenal-derived steroids including DHEA, DHEAS and the 11-oxygenated androgens 11OHA4, 11KA4, 11KT and 11OHT (see FIG. 2).

### Example 2—Clustering Based on 20 Steroids

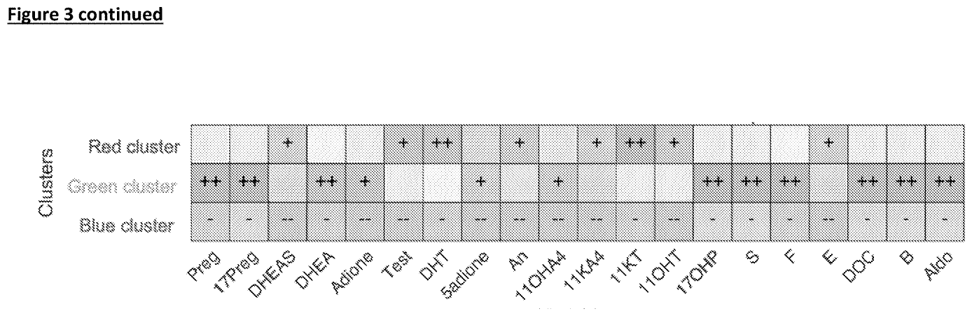

In the clustering with all 20 steroids identified and quantified in the multi-steroid profiling assay, the blue cluster was again characterised by generally lower levels. The green cluster comprises patients with comparably higher glucocorticoid concentrations. The red cluster comprised patients with higher active androgen concentrations, both from the classic and 11-oxygenated androgen pathway, with DHT and 11KT representing the most prominent marker steroids (see FIG. 3).

### Example 3—Cluster Comparison with Healthy Female Controls

The inventors also compared their results to those obtained with healthy female controls, and post hoc visualised them on top of the PCOS patient clusters (see FIG. 4; left panel 11-steroid clustering, right panel 20-steroid clustering—the blue cluster is on the left; the green cluster is on the right and the red cluster is in the middle). This showed that the steroid metabolomes of the healthy controls (indicated in yellow) in the overwhelming majority projected onto the blue cluster.

### CONCLUSION

The inventors propose that this approach can be used to identify PCOS patients with distinct clinical phenotypes, e.g. those at higher metabolic risk and those who respond to certain types of therapy. Thus, the invention helps to provide informed stratified diagnostic and therapeutic decisions, e.g. which patients receive specific enzyme inhibitors or modified release glucocorticoids to suppress the adrenal androgen drive.

Preliminary analysis of the phenotype data for the 11-steroid data set has already shown that the patients in the green cluster, characterised by 11-oxygenated androgens, appear to have a significantly increased metabolic risk, more detailed analysis to follow soon. Our data set is unique in its size and quality, with all patients treatment naïve and fasted when the blood samples were collected, thereby keeping confounding factors/“data noise” to a minimum.

### REFERENCES

1. https://nl.mathworks.com/help/stats/zscore.html
2. https://nl.mathworks.com/help/stats/kmeans.html
3. Lloyd, Stuart P. “Least Squares Quantization in PCM.” IEEE Transactions on Information Theory. Vol. 28, 1982, pp. 129-137.
4. Arthur, David, and Sergi Vassilvitskii. “K-means++: The Advantages of Careful Seeding.” SODA '07: Proceedings of the Eighteenth Annual ACM-SIAM Symposium on Discrete Algorithms. 2007, pp. 1027-1035.

1. A method of stratifying test subjects with Polycystic Ovary Syndrome (PCOS) into those that are expected to respond to therapeutic treatment with an inhibitor of hypothalamic-pituitary-adrenal axis activity, those that are expected to respond to therapeutic treatment with an AKR1C3 inhibitor, and those that are expected to respond to therapeutic treatment with a 5alpha-reductase inhibitor, the method comprising:
(a) providing a test sample from a test subject with PCOS;
(b) determining the concentration/level in the test sample of at least one ovarian androgen steroid and/or a derivative thereof, and at least one adrenal steroid and/or derivative thereof; and
(c) using the results from (b) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

2. The method according to claim 1, wherein the at least one ovarian androgen steroid or derivative thereof is or comprises DHT.

3. The method according to claim 1 or claim 2, wherein the at least one ovarian androgen steroid or derivative thereof is or comprises one or more, two or more, or three or more selected from the group comprising androstenedione (Adione), testosterone, 5α-dihydrotestosterone (DHT) and 5α-androstanedione (5Adione).

4. The method according to claim 3, wherein the at least one ovarian androgen steroid or derivative thereof further comprises androsterone (An).

5. The method according to any one of the preceding claims, wherein the at least one adrenal steroid may be selected from the group comprising dehydroepiandrosterone sulfate (DHEAS), dehydroepiandrosterone (DHEA), 11-hydroxyandrostenedione (11OHA4), 11-ketoandrostenedione (11KA4), 11-ketotestosterone (11KT), 11-hydroxytestosterone (11OHT), pregnenolone, 17-hdyroxypregnenolone, 17-hydroxyprogesterone (17OHP), 11-deoxycortisol (S), cortisol (F), d11-deoxycorticosterone (DOC), corticosterone (B) and aldosterone (Aldo).

6. The method according to claim 1, wherein the at least one the ovarian androgen steroid or derivative thereof is Adione, Test, DHT and 5Adione, and the at least one adrenal steroid or derivative thereof is DHEAS, DHEA, 11OHA4, 11KA4, 11KT and 11OHT.

7. The method according to claim 1, wherein the at least one ovarian androgen steroid or derivative thereof is DHT, and the at least one adrenal steroid or derivative thereof is pregnenolone, 17-hydroxypregnenolone, DHEA, 11KT, 17OHP, S, F, DOC, B and Aldo.

8. A method of stratifying test subjects with PCOS into those that are expected to respond to therapeutic treatment with an inhibitor of hypothalamic-pituitary-adrenal axis activity, those that are expected to respond to therapeutic treatment with an AKR1C3 inhibitor, and those that are expected to respond to therapeutic treatment with a 5alpha-reductase inhibitor, the method comprising:
(a) providing a test sample from a test subject with PCOS;
(b) determining the concentration/level in the test sample of at least one ovarian androgen steroid or derivative thereof comprising DHT, and at least six adrenal steroids and/or derivatives thereof comprising pregnenolone, 17-hydroxypregnenolone, DHEA, 11KT, 17OHP, S, F, DOC, B and Aldo; and
(c) using the results from (b) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

9. The method according to claim 8, wherein step (b) further comprises determining the concentration/level in the test sample of testosterone and/or An.

10. The method according to claim 8, wherein step (b) further comprises determining the concentration/level in the test sample of one or more ovarian androgen steroids and/or the derivatives selected from the group comprising Adione, 5adione, testosterone and An.

11. The method according to any one of claims 8 to 10, wherein step (b) further comprises determining the concentration/level in the test sample of one or more adrenal steroids and/or the derivatives selected from the group comprising DHEAS, Adione, 11OHA4 and 11KA4.

12. A method of stratifying test subjects with PCOS into those that are expected to respond to therapeutic treatment with an inhibitor of hypothalamic-pituitary-adrenal axis activity, those that are expected to respond to therapeutic treatment with an AKR1C3 inhibitor, and those that are expected to respond to therapeutic treatment with a 5alpha-reductase inhibitor, the method comprising:
(a) providing a test sample from a test subject with PCOS;
(b) determining the concentration/level in the test sample of at least three ovarian androgen steroids and/or derivatives thereof comprising testosterone, DHT and 5adione, and at least six adrenal steroids and/or derivatives thereof comprising DHEAS, DHEA, 11OHA4, 11KA4, 11KT and 11OHT; and
(c) using the results from (b) to predict if the subject will respond to therapeutic treatment with an inhibitor of hypothalamic-pituitary-adrenal axis activity, therapeutic treatment with an AKR1C3 inhibitor, or therapeutic treatment with a 5alpha-reductase inhibitor.

13. The method according to claim 12, wherein step (b) further comprises determining the concentration/level in the test sample of at least Adione, and optionally An.

14. The method according to claim 12, wherein step (b) comprises determining the concentration/level in the test sample of at least 10 steroids comprising DHEAS, DHEA, Adione, testosterone, DHT, 5adione, 11OHA4, 11KA4, 11KT and 11OHT.

15. The method according to claim 12, wherein step (b) comprises determining the concentration/level in the test sample of at least 11 steroids comprising DHEAS, DHEA, Adione, testosterone, DHT, 5adione, An, 11OHA4, 11KA4, 11KT and 11OHT.

16. The method according to any one of the preceding claims, wherein the method comprises comparing the concentrations/levels determined in (b) with reference concentrations of the steroids and/or their derivatives thereof from other subjects with PCOS.

17. The method according to claim 16, wherein the step of comparing the concentrations/levels determined in (b) with reference concentrations of the steroids and their derivatives thereof from other subjects with PCOS comprises performing a cluster analysis.
